(19) United States
(12) Patent Application Publication
(10) Pub. No.: US 2008/0081964 A1
(43) Pub. Date: Apr. 3, 2008

(54) METHOD AND APPARATUS FOR DETERMINING HYPERHIDROSIS ZONES

(76) Inventor: Wlodzimierz Paul Zakrzewski, County of Leduc (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/865,667

(22) Filed: Oct. 1, 2007

Related U.S. Application Data
(60) Provisional application No. 60/827,660, filed on Sep. 29, 2006.

(51) Int. Cl. A61B 5/00

(52) U.S. Cl. 600/306

(57) ABSTRACT

A method and an apparatus for determining hyperhidrosis zones of a patient. A flexible sheet carrying a moisture-indicating substance is provided. The moisture indicating substance indicates hyperhidrosis zones, when the moisture-indicating substance is placed against the skin of a patient.

### FIELD

The present application relates to a flexible sheet to be applied to the skin of a patient to determine hyperhidrosis zones.

### BACKGROUND

One treatment of hyperhidrosis that has proven to be effective is botox injections into zones of hyperhidrosis, which correspond to the locations of sweat glands. The location of sweat glands is either determined by a direct observation of the skin by a doctor, or by applying an iodine-starch paste by hand to the target skin. The blackened areas of the iodine-starch past indicate the location of sweat glands. The iodine-starch procedure is messy, stains clothing and, unfortunately, must be repeated at each subsequent appointment.

### SUMMARY

There is provided an apparatus for determining hyperhidrosis zones of a patient, includes a flexible sheet carrying a moisture-indicating substance. The moisture indicating substance indicates hyperhidrosis zones when the moisture-indicating substance is placed against the skin of a patient.

### BRIEF DESCRIPTION OF THE DRAWINGS

These and other features will become more apparent from the following description in which reference is made to the appended drawings. The drawings are for the purpose of illustration only and are not intended to be in any way limiting, wherein:

FIG. 1 is a perspective view of an embodiment of a sweat gland activity delineator consisting of one flexible sheet.

FIG. 2 is a perspective view of another embodiment of a sweat gland activity delineator.

FIG. 3 is a perspective view of the sweat gland activity delineator of FIG. 1 with a cover sheet.

FIG. 4 is a top-down view of an embodiment of a sweat gland activity delineator consisting of two flexible sheets.

FIG. 5 is an exploded view of an embodiment of the sweat gland activity delineator of FIG. 4.

FIG. 6 is an exploded view of a further embodiment of a sweat gland activity delineator consisting of two flexible sheets.

FIG. 7 is a top-down view of the sweat gland activity delineator of FIG. 6.

FIG. 8 is a perspective view of the sweat gland activity delineator of FIG. 5 with a cover sheet.

FIG. 9 is a perspective view of the sweat gland activity delineator of FIG. 5 with a cover sheet, contained in an air-tight and moisture-free pouch.

FIG. 10 is a view of the sweat gland activity delineator of FIG. 5 in use on a human limb.

FIG. 11 is a view of a human arm after the sweat gland activity delineator of FIG. 5 has been applied, used, and removed.

FIG. 12 is a view of the sweat gland activity delineator of FIG. 6 in use on a human limb.

FIG. 13 is a close-up view of several of the aligned perforations from the sweat gland activity delineator of FIG. 6.

### DETAILED DESCRIPTION

Structure and Relationship of Parts:

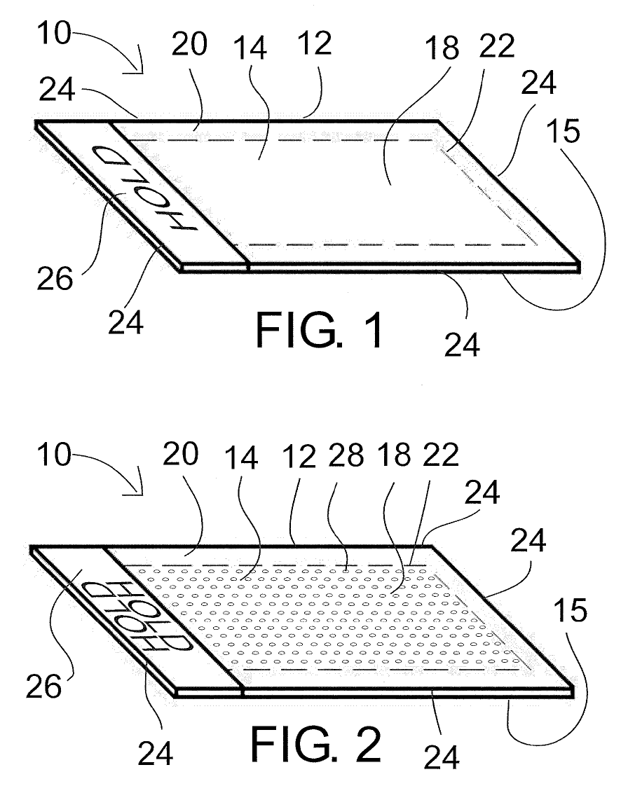

FIG. 1 shows a first embodiment of a sweat gland activity delineator 10, which is a flexible sheet 12 consisting of a first side 14 and a second side 15. First side 14 carries a moisture-indicating substance 18, such that moisture-indicating substance 18 indicates hyperhidrosis zones when placed against the skin of a patient. An adhesive layer 20, adapted to adhere to the skin of a patient, is positioned on first side 14. Adhesive layer 20 is preferably a hypoallergenic adhesive. A margin 22 denotes an area adjacent to side lengths 24 where no moisture indicating substance 18 is placed. In addition, there is a flap 26 having an area adjacent to one or more side lengths 24 of flexible sheet 12 which contains no adhesive layer 20 or moisture indicating substance 18. This allows for easy removal of sweat gland activity delineator 10 from the skin of the patient without contacting moisture indicating substance 18. It will be understood that margin 22 and flap 26 may be modified or removed, depending on the application. As an example, flap 26 of sweat gland activity delineator 10 may contain an area that extends one-inch perpendicularly inward from one of the side lengths 24, while margin 22 has an area extending 0.5 inches perpendicularly inward from the parts of side lengths 24 that are not directly adjacent to flap 26. In the embodiment shown, the word “HOLD” is written on flap 26 to instruct the user to hold flap 26. Alternatively, other words or phrases, or nothing at all may be written on flap 26. Moisture-indicating substance 18 is preferably a starch-iodine mixture, but may be other suitable substances. Adequate results have been obtained with moisture-indicating substance 18 being a mixture of dry iodine powder and dry starch powder. Moisture-indicating substance 18 is carried by or directly adhered to adhesive layer 20. Second side 15 may be smooth, with no adhesive layer 20 or moisture-indicating substance 18.

FIG. 2 shows a second embodiment of a sweat gland activity delineator 10 consisting of a flexible sheet 12. This embodiment contains all the same parts described above for the first embodiment with the addition that flexible sheet 12 contains a series of perforations 28. Perforations 28 allow access to the skin through flexible sheet 12 by a marking instrument 30, such as a pen, as is shown in relation to a further embodiment with respect to FIG. 12. Referring to FIG. 2, the word “HOLD” is written twice on flap 26 to demonstrate an alternative set of words that could be used to instruct the user to hold flap 26.

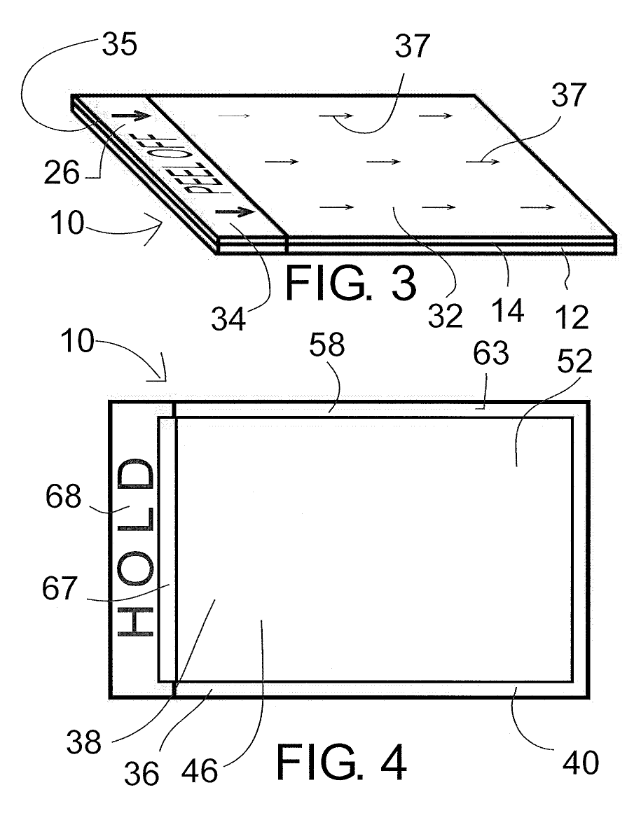

FIG. 3 shows the first embodiment of sweat gland activity delineator 10 wherein first side 14 of flexible sheet 12 is covered by a removable cover sheet 32. Removable cover sheet 32 protects moisture-indicating substance 18 from undesired contact with moisture. It is important that moisture-indicating substance 18 be kept dry in order to work properly. Removable cover sheet 32 contains a cover sheet flap 34. Cover sheet flap 34 has an area adjacent to one end 35 of removable cover sheet 32. End 35 is positioned so that cover sheet flap 34 covers flap 26. Removable cover sheet 32 attaches to flexible sheet 12 by adhering to adhesive layer 20. Because flap 26 contains no adhesive and therefore does not adhere to flap 26, cover sheet flap 34 can be easily gripped. Referring to FIG. 3, the phrase “PEEL OFF” is written on cover sheet flap 34 to instruct the user to remove removable cover sheet 32. This appears on the side of removable cover sheet 32 that is not adhered to flexible sheet 12. Alternatively, other words or phrases, or nothing at all may be written on cover sheet flap 34. Removable cover sheet 32 may also contain arrows 37 that instruct the user to peel off removable cover sheet 32 in the direction indicated. The second embodiment of sweat gland activity delineator 10 operates by the same principles and contains the same parts as described above for the first embodiment. Preferably, sweat gland activity delineator 10 comes packaged in an air-tight, moisture-free pouch before use.

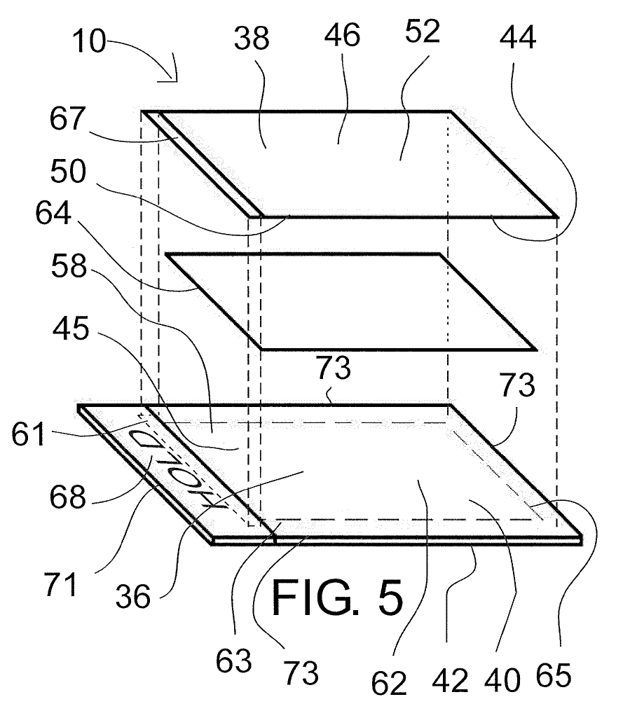

FIG. 5 shows a third embodiment of sweat gland activity delineator 10 consisting of a first flexible sheet 36 and a second flexible sheet 38. First flexible sheet 36 has a first side 40 and a second side 42, and second flexible sheet 38 has a first side 44 and a second side 46. First flexible sheet 36 and second flexible sheet 38 are permeable. First flexible sheet 36 contains a first part 45 of a two part moisture-indicating substance, and second flexible sheet 38 carries a second part 50 of the two part moisture-indicating substance. The two part moisture-indicating substance operates such that when the first part and the second part are brought together in the presence of moisture from the skin of a patient, they act to indicate hyperhidrosis zones. First part 45 consists of one of iodine or starch, and second part 50 consists of the other of iodine or starch. Alternatively, other suitable substances may be used as the two part moisture-indicating substance. Second flexible sheet 38 may be composed of fabric 52 that has had a solution of one of iodine and starch applied and then dried. In the embodiment shown in FIG. 5, fabric 52 has been pre-soaked with a solution of iodine and left to dry.

Referring to FIG. 5, first side 44 of second flexible sheet 38 is positioned up against first side 40 of first flexible sheet 36. In use, second side 46 of second flexible sheet 38 faces the skin of the patient. Positioned on first side 40 of first flexible sheet 36 is an adhesive layer 58, designed to adhere to the skin of a patient, and also to second flexible sheet 38. Adhesive layer 58 is composed of a hypoallergenic adhesive. First part 45 is carried by adhesive layer 58 by adhering first part 45 to an inner portion 62 of adhesive layer 58. An outer portion 63 adheres to both the skin of the patient and second flexible sheet 38. Outer portion 63 is free of first part 45 and second part 50, with a margin 65 separating outer portion 63 from inner portion 62. FIG. 4 shows first flexible sheet 36 and second flexible sheet 38 in contact with each other.

Referring to FIG. 5, a removable impermeable sheet 64 separates first part 45 and second part 50 of the two-part moisture-indicating substance. First part 45 is carried on first flexible sheet 36 and second part 50 is carried on second flexible sheet 38. Removable impermeable sheet 64, such as a thin piece of stiff fabric, prevents first part 45 and second part 50 of the two-part moisture-indicating substance from coming into contact before sweat gland activity delineator 10 is used. Removable impermeable sheet 64 is loosely held between first flexible sheet 36 and second flexible sheet 38 before use, and is not adhered to adhesive layer 58.

As shown in FIG. 8, second side 46 of second flexible sheet 38 is covered by a removable cover sheet 66. Removable cover sheet 66 adheres to outer portion 63 of first flexible sheet 36, and contacts both first flexible sheet 36 and second flexible sheet 38 before its removal. Removable cover sheet 66 protects first part 45 and second part 50 of the two-part moisture-indicating substance from undesired contact with moisture before use. First flexible sheet 36 has a flap 68, which aids in handling sweat gland activity delineator 10. Flap 68 contains no first part 45 or second part 50 of the two-part moisture indicating substance and adhesive layer 58. Removable cover sheet 66 has a cover flap 69, which is positioned over flap 68 of first flexible sheet 36. Flap 68 allows removable cover sheet 66 to be peeled off easily before use, by gripping cover flap 69 with fingers. Flap 68 also allows sweat gland activity delineator 10 to be peeled off of the skin of the patient easily after use. In the embodiment shown in FIG. 5, flap 68 contains an area that extends one-inch perpendicularly outwards from an end 71 of first flexible sheet 36. In addition, outer portion 63 contains an area that extends 0.5 inches perpendicularly outwards from one or more side lengths 73 of first flexible sheet 36. Second flexible sheet 38 may also have a flap 67, without either of first part 45 or second part 50 of the two-part moisture-indicating substance. Flap 67 covers a portion 61 of flap 68. Also shown in the embodiment of FIG. 5, inner portion 62 contains powderized starch adhered to adhesive layer 58.

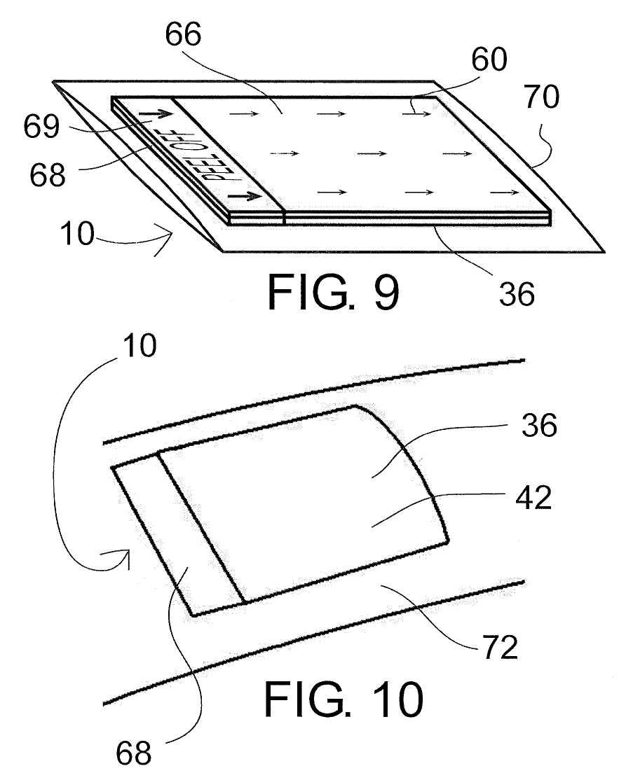

The word “HOLD” may be written on flap 68, as shown in FIGS. 4 and 5. This is written to instruct the user to hold first flexible sheet 36 by flap 68. Alternatively, other words or phrases may be written on flap 68. The words “PEEL-OFF” may be written on cover flap 69, as shown in FIG. 8. This is written to instruct the user to peel-off removable cover sheet 66 by flap 68. Alternatively, other words or phrases may be written on cover flap 69. In addition, arrows 60 appear on removable cover sheet 66, to instruct the user to peel in the direction indicated. Referring to FIG. 9, sweat gland activity delineator 10 with attached removable cover sheet 66 comes contained in a sealed moisture-free pouch 70.

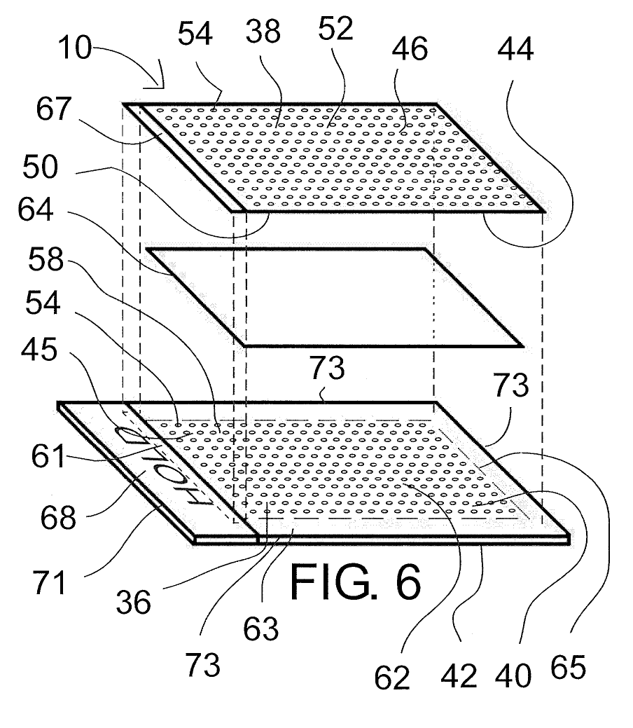

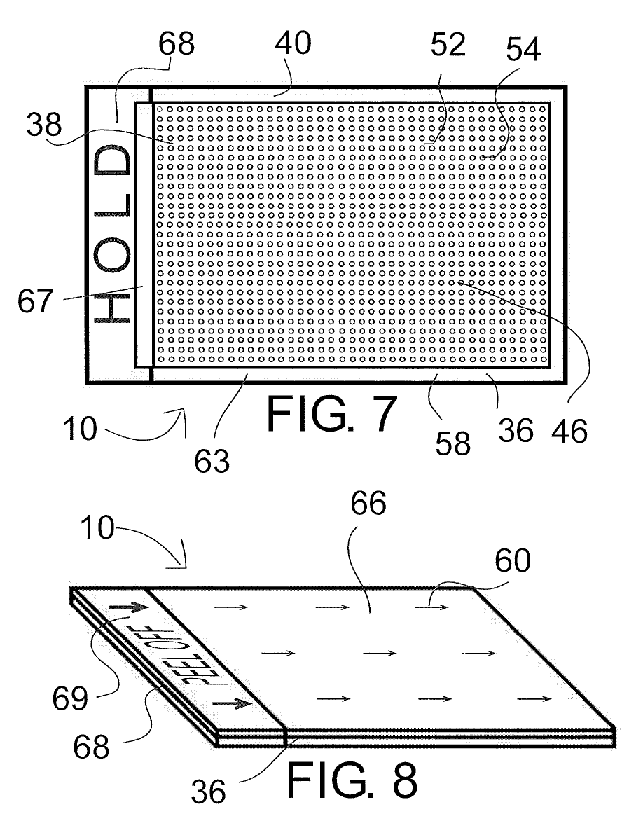

FIG. 6 shows a fourth embodiment of sweat gland activity delineator 10 consisting of a first flexible sheet 36 and a second flexible sheet 38. This embodiment contains all the same parts described above for the third embodiment with the addition that both first flexible sheet 36 and second flexible sheet 38 contain a series of aligned perforations 54. Aligned perforations 54 allow access to the skin through first flexible sheet 36 and second flexible sheet 38 by a marking instrument 30, such as a pen, as is described in relation to a further embodiment with respect to FIG. 12. FIG. 7 shows first flexible sheet 36 and second flexible sheet 38 of the fourth embodiment of a sweat gland activity delineator 10 in contact with each other.

FIG. 10 shows the third embodiment of sweat gland activity delineator 10 in use on a skin surface 72 of a human patient. The first embodiment of sweat gland activity delineator 10, operates by the same principles. Removable impermeable sheet 64 and removable cover sheet 66 have both been removed.

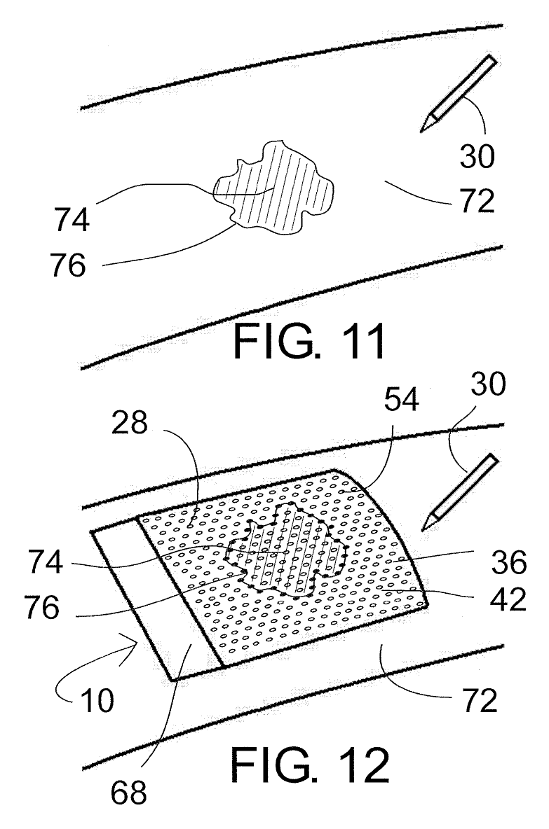

FIG. 11 shows skin surface 72 from FIG. 10, after the third embodiment of sweat gland activity delineator 10 has been removed. The presence of sweat on skin surface 72 has moistened the dehydrated iodine and starch particles from sweat gland activity delineator 10, and formed a dark blue complex. This complex has stained skin surface 72 dark blue over a zone of hyperhidrosis 74. Zone of hyperhidrosis 74 is delineated by outline 76. Outline 76 consists of markings made with marking instrument 30.

FIG. 12 shows the fourth embodiment of sweat gland activity delineator 10 in use on a skin surface 72 of a human patient. The second embodiment of sweat gland activity delineator 10 operates by the same principles. Removable impermeable sheet 64 and removable cover sheet 66 have both been removed. A zone of hyperhidrosis 74 is delineated by outline 76, consisting of markings made with marking instrument 30, made through aligned perforations 54.

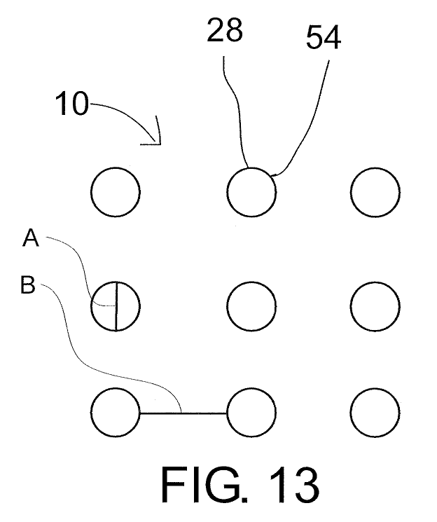

FIG. 13 shows the hole spacing of aligned perforations 54 of the fourth embodiment of a sweat gland activity delineator 10. Each perforation 54 shown has a diameter A of 3 millimetres. Individual perforations are separated by a distance B of 7 millimetres. The values for diameter A and distance B may differ from those described above, as long as both are consistent for any given sweat gland activity delineator 10. The hole spacing described above for aligned perforations 54 are the same for series of perforations 28 of flexible sheet 12 from the second embodiment.

### OPERATION OF THE FIRST EMBODIMENT

The first embodiment of sweat gland activity delineator 10 is removed from the sealed, moisture-free package it comes contained within, similar to pouch 70 shown in FIG. 9. Referring to FIG. 3, cover sheet flap 34 is gripped with fingers, and removable cover sheet 32 is peeled off of first side 14 of flexible sheet 12. Immediately, first side 14 of flexible sheet 12 is applied directly to skin surface 72 over a suspected zone of hyperhidrosis 74, as shown in FIG. 10 for the third embodiment of sweat gland activity delineator 10. Light pressure is applied, which ensures good contact between skin surface 72 and sweat gland activity delineator 10. The presence of sweat on skin surface 72 moistens the dehydrated iodine and starch particles, forming a dark blue complex. This complex stains skin surface 72 and flexible sheet 12 dark blue over zone of hyperhidrosis 74. Sweat gland activity delineator 10 is held in contact with skin surface 72 for a sufficient amount of time. Gripping flap 26 with fingers, flexible sheet 12 is peeled off of skin surface 72 and disposed. Referring to FIG. 11, outline 76 is then drawn directly around zone of hyperhidrosis 74, as indicated by dark staining present on skin surface 72. Outline 76 is made using marking instrument 30. After outline 76 is drawn, skin surface 72 is cleaned with moist gauze to remove any stain present as a result of the moisture-indicating substance 18. Botox may then be applied to zone of hyperhidrosis 74, in order to treat hyperhidrosis. If desired, a map may be made of zones of hyperhidrosis 74 for future treatments.

### OPERATION OF THE SECOND EMBODIMENT

The second embodiment of sweat gland activity delineator 10 is removed from the sealed, moisture-free package it comes contained within, similar to pouch 70 shown in FIG. 9. Referring to FIG. 3, cover sheet flap 34 is gripped with fingers, and removable cover sheet 32 is peeled off of first side 14 of flexible sheet 12. Immediately, first side 14 of flexible sheet 12 is applied directly to skin surface 72 over a suspected zone of hyperhidrosis 74, as shown in FIG. 12 for the fourth embodiment of sweat gland activity delineator 10. Light pressure is applied, which ensures good contact between skin surface 72 and sweat gland activity delineator 10. The presence of sweat on skin surface 72 moistens the dehydrated iodine and starch particles, forming a dark blue complex. This complex stains skin surface 72 and flexible sheet 12 dark blue over zone of hyperhidrosis 74. Sweat gland activity delineator 10 is held in contact with skin surface 72 for a sufficient amount of time. An outline 76 of zone of hyperhidrosis 74 is then made using marking instrument 30, through series of perforations 28. Gripping flap 26 with fingers, flexible sheet 12 is peeled off of skin surface 72 and disposed. After flexible sheet 12 is removed, skin surface 72 is cleaned with moist gauze to remove any stain present as a result of the moisture-indicating substance 18. Botox may then be applied to zone of hyperhidrosis 74, in order to treat hyperhidrosis. If desired, a map may be made of zones of hyperhidrosis 74 for future treatments.

### OPERATION OF THE THIRD EMBODIMENT

Referring to FIG. 9, the third embodiment of sweat gland activity delineator 10 is removed from sealed moisture-free pouch 70 it comes contained within. Gripping cover flap 69 with fingers, removable cover sheet 66 is peeled off of first flexible sheet 36. Immediately, sweat gland delineator 10 is applied directly to skin surface 72, as shown in FIG. 12, with second side 46 of second flexible sheet 38 below first flexible sheet 36 (as shown in FIG. 5) contacting skin surface 72 over a suspected zone of hyperhidrosis 74. Removable impermeable sheet 64, shown in FIG. 5, is carefully removed, and light pressure is applied, which ensures good contact between skin surface 72 and sweat gland delineator 10. Referring still to FIG. 12, the presence of moisture on skin surface 72 moistens second part 50 of the two-part moisture-indicating substance, and permeates second part 50 into contact with first part 45, contained on first flexible sheet 36, forming a dark blue complex. This complex stains skin surface 72, first flexible sheet 36, and second flexible sheet 38 dark blue over zone of hyperhidrosis 74. Sweat gland activity delineator 10 is held in contact with skin surface 72 for a sufficient amount of time. Gripping flap 68 with fingers, sweat gland delineator 10 is peeled off of skin surface 72 and disposed of. Outline 76 is drawn directly around zone of hyperhidrosis 74, as indicated by dark blue staining present on skin surface 72, using marking instrument 30. After outline 76 has been drawn, skin surface 72 is cleaned with moist gauze to remove any stain present as a result of the two-part moisture-indicating substance. Botox may then be applied to zone of hyperhidrosis 74, in order to treat zone of hyperhidrosis 74.

### OPERATION OF THE FOURTH EMBODIMENT

Referring to FIG. 9, the fourth embodiment of sweat gland activity delineator 10 is removed from sealed moisture-free pouch 70 it comes contained within. Gripping cover flap 69 with fingers, removable cover sheet 66 is peeled off of first flexible sheet 36. Immediately, sweat gland delineator 10 is applied directly to skin surface 72, as shown in FIG. 12, with second side 46 of second flexible sheet 38 below first flexible sheet 36 (as shown in FIG. 7) contacting skin surface 72 over a suspected zone of hyperhidrosis 74. Removable impermeable sheet 64, shown in FIG. 6, is carefully removed, and light pressure is applied, which ensures good contact between skin surface 72 and sweat gland delineator 10. Referring still to FIG. 12, the presence of moisture on skin surface 72 moistens second part 50 of the two-part moisture-indicating substance, and permeates second part 50 into contact with first part 45, contained on first flexible sheet 36, forming a dark blue complex. This complex stains skin surface 72, first flexible sheet 36, and second flexible sheet 38 dark blue over zone of hyperhidrosis 74. Sweat gland activity delineator 10 is held in contact with skin surface 72 for a sufficient amount of time. Outline 76 of zone of hyperhidrosis 74 is then made using marking instrument 30, through aligned perforations 54. Gripping flap 68 with fingers, sweat gland delineator 10 is peeled off of skin surface 72 and disposed of. After outline 76 has been drawn, skin surface 72 is cleaned with moist gauze to remove any stain present as a result of the two-part moisture-indicating substance. Botox may then be applied to zone of hyperhidrosis 74, in order to treat zone of hyperhidrosis 74.

### ADVANTAGES

The teachings of the present invention provide an approach which speeds up the location of hyperhidrosis zones and makes the process less messy. It also provides an accurate “map” that can be used to provide a guide for use in locating hyperhidrosis zones on subsequent visits. This guide can also be used with methods used to treat the symptoms of hyperhidrosis, such as botox injection, by outlining the areas which require treatment.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

It will be apparent to one skilled in the art that modifications may be made to the illustrated embodiment without departing from the spirit and scope defined in the Claims.

What is claimed is:

1. An apparatus for determining hyperhidrosis zones of a patient, comprising:
a flexible sheet carrying a moisture-indicating substance, such that the moisture indicating substance indicates hyperhidrosis zones when the moisture-indicating substance is placed against the skin of a patient.

2. The apparatus of claim 1, wherein the flexible sheet includes an adhesive layer adapted to adhere to skin of a patient, the adhesive layer being positioned on a first side of the flexible sheet.

3. The apparatus of claim 2, wherein the moisture-indicating substance is carried by the adhesive layer.

4. The apparatus of claim 3, wherein the moisture-indicating substance is adhered to the adhesive layer.

5. The apparatus of claim 1, wherein the moisture-indicating substance comprises a starch iodine mixture.

6. The apparatus of claim 1, wherein the flexible sheet has a series of perforations to allow access to the skin through the flexible sheet by a marking instrument.

7. The apparatus of claim 2, wherein the first side is covered by a removable cover sheet, the removable cover protecting the moisture-indicating substance from undesired contact with moisture.

8. The apparatus of claim 7, wherein the flexible sheet has a flap to aid in handling the flexible sheet, the flap being free of the moisture indicating substance and adhesive.

9. An apparatus for determining hyperhidrosis zones of a patient, the apparatus comprising:
a moisture-indicating substance comprising a first part and a second part, such that the first part and the second part combine and undergo a chemical reaction in the presence of moisture;
a first flexible sheet having a first side, the first side carrying the first part of the moisture-indicating substance;
a second flexible sheet having a first side and a second side, the second flexible sheet being permeable, the second flexible sheet carrying the second part of the moisture-indicating substance, the first side of the first flexible sheet being placed against the first side of the second flexible sheet, and the second side of the second flexible sheet is placed against the skin of the patient, such that moisture from the skin of the patient activates the first part and the second part of the moisture-indicating substance to indicate hyperhidrosis zones on the skin of the patient.

10. The apparatus of claim 9, wherein the first part of the moisture-indicating substance comprises one of iodine or starch, and the second part of the moisture-indicating substance comprises the other of iodine or starch.

11. The apparatus of claim 10, wherein the second flexible sheet comprises fabric that carries a dried solution of one of iodine and starch.

12. The apparatus of claim 11, wherein the first flexible sheet is attached to the second flexible sheet.

13. The apparatus of claim 12, wherein the first part of the moisture-indicating substance, carried on the first flexible sheet, and the second part of the moisture-indicating substance, carried on the second flexible sheet, are separated by a removable impermeable sheet prior to the moisture-indicating substance being activated.

14. The apparatus of claim 12, wherein the first flexible sheet is attached to the second flexible sheet by an adhesive perimeter on the first flexible sheet.

15. The apparatus of claim 14, wherein the adhesive perimeter extends beyond the second flexible sheet and attaches to the skin of the patient.

16. The apparatus of claim 9, wherein the first side of the first flexible sheet has an adhesive layer.

17. The apparatus of claim 16, wherein an outer portion of the adhesive layer adheres to the skin of the patient and to the second flexible sheet, the first part of the moisture-indicating substance being carried by an inner portion of the adhesive layer.

18. The apparatus of claim 9, wherein the first flexible sheet and the second flexible sheet have a series of aligned perforations to allow access to the skin through the first flexible sheet and the second flexible sheet by a marking instrument.

19. The apparatus of claim 9, wherein the second side of the second flexible sheet is covered by a removable cover sheet, the removable cover sheet protecting the moisture-indicating substance from undesired contact with moisture.

20. The apparatus of claim 9, wherein the flexible sheet has a flap to aid in handling the flexible sheet, the flap being free of the moisture indicating substance and adhesive.

21. A method for determining hyperhidrosis zones of a patient, comprising the steps of:
providing an apparatus for determining hyperhidrosis zones, the apparatus comprising at least one flexible sheet carrying a moisture-indicating substance;
placing the apparatus against the skin of a patient and allowing the apparatus to be exposed to moisture excreted by the patient until moisture from the patient reacting with the moisture-indicating substance identifies the patient's hyperhidrosis zones.

22. The method as defined in claim 21, including a further step of marking the patient's hyperhidrosis zones.
